(19) United States
(12) Patent Application Publication
(10) Pub. No.: US 2023/0146331 A1
(43) Pub. Date: May 11, 2023

(54) Pharmaceutical Composition Made on Plant Raw Materials for Treating and Preventing Cancer

(71) Applicants: Babry Oren, Van Nuys, CA (US); Sasha Moshe, Alpharetta, GA (US)

(72) Inventors: Babry Oren, Van Nuys, CA (US); Sasha Moshe, Alpharetta, GA (US)

(21) Appl. No.: 18/149,101

(22) Filed: Dec. 31, 2022

(51) Int. Cl. A61K 36/15; A61K 36/87; A61K 36/906; A61K 36/9066; A61K 36/65; A61K 36/41; A61K 36/736; A61K 36/07; A61K 36/28; A61K 36/16; A61K 36/67; A61K 36/61; A61K 36/73; A61K 31/525

(52) U.S. Cl. CPC A61K 36/15; A61K 31/525; A61K 36/07; A61K 36/16; A61K 36/28; A61K 36/41; A61K 36/61; A61K 36/65; A61K 36/67; A61K 36/73; A61K 36/87; A61K 36/736; A61K 36/906; A61K 36/9066

(57) ABSTRACT

A pharmaceutical composition containing dry powder of pine (Pinus) needles and dry peels and pits of grapes extract and pharmaceutically acceptable additive, dry extracts of ginger (Zingiber officinale), turmeric (Curcuma longa), white peony (Paeonia), Rhodiola Rosea (Rhodiola Rosea), apricot (Prunus) seed, oyster mushroom (Pleurotus ostreatus), green tea (Camellia sinensis), Ginkgo Biloba (Ginkgo biloba), white pepper, pomegranate (Punica granatum) pulp, medlar (Mespilus germanica), dry powder of pomegranate (Punica granatum) juice and folic acid, in the following ratio of the components in weight % (w %):

| | |
|---|---|
| dry powder of pine needles and dry peels and pits of grapes extract and pharmaceutically acceptable additive | 6-24 |
| dry extract of ginger | 6-25 |
| dry extract of turmeric | 5-24 |
| dry extract of white peony | 2-20 |
| dry extract of Rhodiola Rosea | 2-20 |
| dry extract of apricot seed | 5-20 |
| dry extract of oyster mushroom | 10-34 |
| dry extract of green tea | 12-35 |
| dry extract of Ginkgo Biloba | 2-25 |
| dry extract of white pepper | 1-15 |
| dry extract of pomegranate pulp | 2-20 |
| extract of medlar | 2-20 |
| dry powder of pomegranate juice | 2-25 |
| folic acid | 3-30 |

### TECHNICAL FIELD

The invention relates to the pharmaceutical industry and applies to pharmaceutical compositions made on plant raw materials, which can be used for treating cancer.

### BACKGROUND ART

Cancer is a disease that affects millions of people worldwide every year. Cancer has been one of the ten leading causes of death during the last 30 years. The main cause of cancer is the abnormality of cells, which are constantly dividing and thus producing more and more abnormal cells.

The cancer refers to a family of diseases that starts mainly from the uncontrolled proliferation of cells, invades neighboring normal tissues or organs and establishes a new growth place therein and that can eventually take an individual’s life. In the recent period, significant successes have been achieved in regulation of cell cycle and apoptosis. Also, novel targets are in the process of development including oncogenes and tumor suppressor genes. In spite of these efforts, the incidence of cancer is increasing worldwide, especially in developed countries.

At present, treatment of cancer patients includes surgical interventions, radiotherapy and chemotherapy, which for its part involves more than 40 anticancer substances having strong cytotoxicity.

Chemotherapy, which involves administering anticancer substances into organism, has been widely used in the treatment of different types of cancer. However, chemotherapy is often not effective, especially in treatment of colon and lung cancer. This is mainly due to the resistance of cancer cells to several anticancer agents.

Most currently used anticancer agents were developed based on the fact that cancer cells divide more rapidly than normal cells. Therefore, compounds have been developed which contain inhibitors of DNA replication or synthesis, metabolism inhibitors, cell division inhibitors, nucleotide analogues and topoisomerase inhibitors. Thus, anticancer agents affect the division and survival rate of cells.

The effects of anticancer agents on cancer cells can be described with respect to the following three aspects: apoptosis of cancer cells, migration of cancer cells and cancer metastasis.

In the abovementioned aspects one of the most important is apoptosis. All cells of a multicellular organism have a potential to induce apoptosis, and hemostasis is maintained by the growth and death of cells. In an adult, 50-70 billion cells are destroyed daily by an apoptosis process and a similar number of cells are generated, thus maintaining homeostasis. Cells undergoing apoptosis are absorbed by phagocytosis by surrounding cells. Under normal conditions, apoptosis involves the destruction of cancer cells. However, since cancer cells have a defect in signal associated with apoptosis, they increase in number and have resistance to anticancer agents.

Critical proteins involved in apoptosis have been conserved during animal evolution and have become targets of viruses. The evolutionary conserved proteins are key factors in apoptosis pathways. Mentioned proteins are caspase/CED3, Apaf-1/CED and Bcl-2/CED-9.

Apoptosis is triggered mainly by two signaling pathways: 1) death signal receptors present in the plasma membrane, and 2) mitochondria signal of cells.

Cell death receptors belong to the tumor necrosis factor (TNF) family and is characterized by having an intercellular death domain. The death domain binds to FADD (fas-associated death domain protein), then FADD binds to inactive caspase-8 and caspase-10, resulting in caspase activation by self-cleavage.

Mitochondria-dependent apoptosis signaling is initiated by damaging mitochondria membrane and as a result, inducing the release of cytochrome and other death factors from mitochondria. Cytochrome c in cytosol binds APAF1 (activating factor of apoptotic protease -1), ATP and procaspase-9 to trigger the activation of caspase-9. Activated caspases amplify death cell signaling by cleaving other inactive caspases. Eventually, activated caspases degrade cell death substrates, resulting in the characteristic morphological and biochemical properties of death cells. Representative examples of the cell death substances are as follows: the degradation of lamins induces nuclear shrinkage, the degradation of PARP (poly(ADP-ribose)polymerase) results in the suppression of restoration of DNA damaged by external stresses, leading to cell death. In addition, cells shrink as a result of cellular skeleton proteins degradation and then they are absorbed by macrophages.

Stresses, such as oncogenes, DNA damage, hypoxia and starvation, trigger the death of cells. In the mentioned process, one of the critical regulators is p53. p53 stimulates the expression of Bax, Bak, Puma and Noxa, which promote apoptosis, while inhibiting the Bcl-2 activity of survival factor. Cancer cells often have defects in intrinsic apoptosis pathways. For example, in more than 50% cases the p53 suppressor gene is present in a mutated form. Also, defects are found in p53 mediators, such as PTEN, Bax, Bak and Apaf-1, in p53 regulators, such as ATM, Chk2, Mdm2 and p19ARF, as a result, these defects inhibit apoptosis inducing p53 (Nature Review drug Discovery 2002, 1, p.111-121).

Since anticancer agents, as mentioned above, mainly affect cell division, their use is problematic due to their toxicity. In addition, anticancer agents have another major drawback, which is that they affect rapidly dividing normal cells. Examples of such cells are bone marrow cells and intestinal epithelial cells. Consequently, the development of less toxic anticancer agents is an urgent task. Therefore, the use of herbal preparations against cancer has recently become relevant, as they are considered to be the means causing less side effects.

In spite of the considerable number of medicinal products based on plant raw materials, it is still relevant to develop such herbal remedies that will affect the above-described apoptosis factors, therefore, will be effective in the treatment of cancer and will also be safe and with fewer side effects.

Antioxidant, radioprotective, lipid metabolism regulating and cardiovascular disease medicinal preparation (GE5361 (Vazha Khositashvili, Levan Khositashvili, Babry Oren) 26.12.2011) is known, which contains extract of pine (Pinus) needles and dry peels and pits of grapes and a pharmaceutical additive, mainly, sucrose. However, this preparation does not have an anti-cancer effect.

### BRIEF DISCLOSURE OF THE INVENTION

One object of the invention is the pharmaceutical composition based on plant raw materials, which contains dry powder of pine (Pinus) needles and dry peels and pits of grapes extract and pharmaceutically acceptable additive, dry extracts of ginger (Zingiber officinale), turmeric (Curcuma longa), white peony (Paeonia), Rhodiola Rosea (Rhodiola Rosea), apricot (Prunus) seed, oyster mushroom (Pleurotus ostreatus), green tea (Camellia sinensis), Ginkgo Biloba (Ginkgo Biloba), white pepper, pomegranate (Punica granatum) pulp, medlar (Mespilus germanica), dry powder of pomegranate (Punica granatum) juice and folic acid, in the following ratio of the components in weight % (w %):

| | |
|---|---|
| dry powder of pine needles and dry peels and pits of grapes extract and pharmaceutically acceptable additive | 6-24 |
| dry extract of ginger | 6-25 |
| dry extract of turmeric | 5-24 |
| dry extract of white peony | 2-20 |
| dry extract of Rhodiola Rosea | 2-20 |
| dry extract of apricot seed | 5-20 |
| dry extract of oyster mushroom | 10-34 |
| dry extract of green tea | 12-35 |
| dry extract of Ginkgo Biloba | 2-25 |
| dry extract of white pepper | 1-15 |
| dry extract of pomegranate pulp | 2-20 |
| extract of medlar | 2-20 |
| dry powder of pomegranate juice | 2-25 |
| folic acid | 3-30 |

In the preferable version of the invention embodiment the composition contains components in the following ratio in w%:

| | |
|---|---|
| dry powder of pine needles and dry peels and pits of grapes extract and pharmaceutically acceptable additive | 7 |
| dry extract of ginger | 8 |
| dry extract of turmeric | 10 |
| dry extract of white peony | 12 |
| dry extract of Rhodiola Rosea | 10 |
| dry extract of apricot seed | 7 |
| dry extract of oyster mushroom | 10 |
| dry extract of green tea | 12 |
| dry extract of Ginkgo Biloba | 7 |
| dry extract of white pepper | 3 |
| dry extract of pomegranate pulp | 3 |
| extract of medlar | 5 |
| dry powder of pomegranate juice | 3 |
| folic acid | 3 |

Another object of the invention is a medicament which contains the above mentioned composition.

In the preferable version of the invention embodiment the medicament has a form of a capsule.

In the preferable version of the invention embodiment the medicament contains the composition in the amount of 400-480 mg, in more preferable version in the amount of 450 mg.

### FULL DISCLOSURE OF THE INVENTION

One object of the invention is the pharmaceutical composition, which contains dry powder of pine (Pinus) needles and dry peels and pits of grapes extract and pharmaceutically acceptable additive, dry extracts of ginger (Zingiber officinale), turmeric (Curcuma longa), white peony (Paeonia), Rhodiola Rosea (Rhodiola Rosea), apricot (Prunus) seed, oyster mushroom (Pleurotus ostreatus), green tea (Camellia sinensis), Ginkgo Biloba (Ginkgo biloba), white pepper, pomegranate (Punica granatum) pulp, medlar (Mespilus germanica), dry powder of pomegranate (Punica granatum) juice and folic acid, in the following ratio of the components in w %:

| | |
|---|---|
| dry powder of pine needles and dry peels and pits of grapes extract and pharmaceutically acceptable additive | 6-24 |
| dry extract of ginger | 6-25 |
| dry extract of turmeric | 5-24 |
| dry extract of white peony | 2-20 |
| dry extract of Rhodiola Rosea | 2-20 |
| dry extract of apricot seed | 5-20 |
| dry extract of oyster mushroom | 10-34 |
| dry extract of green tea | 12-35 |
| dry extract of Ginkgo Biloba | 2-25 |
| dry extract of white pepper | 1-15 |
| dry extract of pomegranate pulp | 2-20 |
| extract of medlar | 2-20 |
| dry powder of pomegranate juice | 2-25 |
| folic acid | 3-30 |

As a result of long-term experimental studies, inventors have found that the components in the composition have a synergistic effect, in terms of anticancer effect, which is most likely due to their combined impact on apoptosis regulatory factors.

The composition is prepared as follows: initially, the components included in the composition are prepared separately. Extract of pine needles and dry peels and pits of grapes is prepared according to the method described in Georgian Patent GE5361. Liquid pharmaceutically acceptable additive is added to the obtained extract, preferably sucrose. The extract and the pharmaceutically acceptable additive are mixed in the same ratio as described in Georgian Patent GE5361. Finally obtained mixture is dried till making a dry powder. Drying is possible by any method known in the pharmaceutical industry, preferably spray drying is used. Extracts of ginger, turmeric, white peony, Rhodiola Rosea, apricot seed, oyster mushroom, green tea, Ginkgo Biloba, white pepper, pomegranate pulp, medlar are prepared separately. Extracts are prepared by any technology known in the pharmaceutical industry. Obtained liquid extracts are dried separately. Drying is possible by any method known in the pharmaceutical industry, preferably spray drying is used. Finally, dry extracts are obtained. Pomegranate juice is obtained by any known technology, preferably by pressing. Obtained juice is dried. Drying is possible by any method known in the pharmaceutical industry, preferably spray drying is used. Finally, dry powder is obtained. The powders obtained separately by the method described above are mixed together until a homogeneous mass is obtained, after which folic acid is added and stirring continuous. The components are mixed in such a ratio that the finally obtained composition contains ingredients in the following ratio in w%:

| | |
|---|---|
| dry powder of pine needles and dry peels and pits of grapes extract and pharmaceutically acceptable additive | 6-24 |
| dry extract of ginger | 6-25 |
| dry extract of turmeric | 5-24 |
| dry extract of white peony | 2-20 |
| dry extract of Rhodiola Rosea | 2-20 |
| dry extract of apricot seed | 5-20 |
| dry extract of oyster mushroom | 10-34 |
| dry extract of green tea | 12-35 |
| dry extract of Ginkgo Biloba | 2-25 |
| dry extract of white pepper | 1-15 |
| dry extract of pomegranate pulp | 2-20 |
| extract of medlar | 2-20 |
| dry powder of pomegranate juice | 2-25 |
| folic acid | 3-30 |

One more object of the invention is a medicament. In preferable version of the embodiment of the invention the medicament has a form of a capsule. In order to obtain the medicament in a form of a capsule, gelatinous capsules are filled with the above mentioned composition, by the method well-known in the pharmaceutical industry. In preferable version of the invention embodiment the capsule contains the composition in the amount of 400-480 mg, in more preferable version in the amount of 450 mg.

Indications for using the medicament are as follows: treatment and prevention of cancer of different localization.

Dosage of the medicament (preferably capsule) is 400-480 mg (one capsule) 2-3 times per day. Peroral administration of the medicament is possible, though it is better to dissolve the powder contained in the medicament (for example a capsule) in 32° C. preboiled water and take it orally in a form of liquid. The medicament is administered 15-30 minutes before eating.

### Specific Examples of Carrying Out of the Invention

### Example 1

The composition contains the components in the following ratio in mg:

| | |
|---|---|
| dry powder of pine needles and dry peels and pits of grapes extract and pharmaceutically acceptable additive | 31.5 |
| dry extract of ginger | 36 |
| dry extract of turmeric | 45 |
| dry extract of white peony | 54 |
| dry extract of Rhodiola Rosea | 45 |
| dry extract of apricot seed | 31.5 |
| dry extract of oyster mushroom | 45 |
| dry extract of green tea | 54 |
| dry extract of Ginkgo Biloba | 31.5 |
| dry extract of white pepper | 13.5 |
| dry extract of pomegranate pulp | 13.5 |
| extract of medlar | 22.5 |
| dry powder of pomegranate juice | 13.5 |
| folic acid | 13.5 |
| total mass of the composition | 450 mg |

### Example 2

The composition contains the components in the following ratio in mg:

| | |
|---|---|
| dry powder of pine needles and dry peels and pits of grapes extract and pharmaceutically acceptable additive | 28 |
| dry extract of ginger | 32 |
| dry extract of turmeric | 40 |
| dry extract of white peony | 48 |
| dry extract of Rhodiola Rosea | 40 |
| dry extract of apricot seed | 28 |
| dry extract of oyster mushroom | 40 |
| dry extract of green tea | 48 |
| dry extract of Ginkgo Biloba | 28 |
| dry extract of white pepper | 12 |
| dry extract of pomegranate pulp | 12 |
| extract of medlar | 20 |
| dry powder of pomegranate juice | 12 |
| folic acid | 12 |
| total mass of the composition | 400 mg |

### Example 3

The composition contains the components in the following ratio in mg:

| | |
|---|---|
| dry powder of pine needles and dry peels and pits of grapes extract and pharmaceutically acceptable additive | 34 |
| dry extract of ginger | 39 |
| dry extract of turmeric | 44 |
| dry extract of white peony | 53 |
| dry extract of Rhodiola Rosea | 45 |
| dry extract of apricot seed | 35 |
| dry extract of oyster mushroom | 48 |
| dry extract of green tea | 58 |
| dry extract of Ginkgo Biloba | 34 |
| dry extract of white pepper | 17.5 |
| dry extract of pomegranate pulp | 17.5 |
| extract of medlar | 20 |
| dry powder of pomegranate juice | 17.5 |
| folic acid | 17.5 |
| total mass of the composition | 480 mg |

A number of researches have been carried out to study the effectiveness of the composition of the invention.

### Example 1 (Effect on Protein P53 Expression)

Human lung cancer cells A549 were treated with the aqueous solution of the composition described in Example 1 at a concentration of 4 mg/ml for 24 hours, and as a control, lung cancer cells A549 were treated with pure water for 24 hours. Then, in both samples, p53 protein expression was determined by western blotting, which was three times higher than the control.

### Example 2 (Effect on Apoptosis)

Human lung cancer cells A549 were treated with the aqueous solutions of the composition described in Examples 1-3, with a concentration of 4 mg/ml for 72 hours, and survival rates of the cells were determined by an MTT assay. As a control, human lung cancer cells A549 were treated with pure water for 72 hours. The results of the study are shown in Table 1.

TABLE 1

| Composition Version | Survival Rate % |
|---|---|
| Example 1 | 5 |
| Example 2 | 7 |
| Example 3 | 12 |

Survival rate % = average absorbance of experimental group (compositions)/average absorbance of control

### Example 3 (Effect on the Cell Cycle G1)

Human lung cancer cells A549 were treated with the aqueous solution of the composition described in Example 1 at a concentration of 4 mg/ml for 24 hours. Cells were then stained with propidium iodide and cell cycle G1 was determined by flowing out cytometry. As a result of the study, it was found that the number of cells with arrested G1 cycle, increased to 86.4%, while in the control (lung cancer cells A549 were treated with pure water) this index was 54.8%.

### Example 4 (Study of the Anti-Cancer Effect of the Composition)

Human colon cancer cells SW620 were used for the cancer line. A suspension of the mentioned cancer cells (1×10⁷ cell/ml) was administered to 40 mice subcutaneously, in amount of 0.3 ml/mouse. The mice were divided in 4 groups. The mice of the first group (study group) were given the aqueous solution of the composition described in Example 1 orally, with 5 mg/kg dosage of the active ingredient once a day for 20 days after the cancer cell transplantation. The mice of the second group (study group) were given the aqueous solution of the composition described in Example 2 orally, with 5 mg/kg dosage of the active ingredient once a day for 20 days after the cancer cell transplantation. The mice of the third group (study group) were given the aqueous solution of the composition described in Example 3, with 5 mg/kg dosage of the active ingredient once a day for 20 days after the cancer cell transplantation. The mice of the fourth group (control group) were given a 0.5% aqueous solution of Twin 80 with 10 ml/kg dosage once a day for 20 days after the cancer cell transplantation.

On days 8, 11, 14, 17, 20, and 22 after the cancer cell transplantation, tumor size was measured. On days 8, 10, 12, 14, 16, 18, 20 and 22 after the cancer cell transplantation, the weight of the mice was determined.

On day 22 after the cancer cell transplantation, all mice were killed, tumors were isolated and weighed.

There was no weight loss of mice in any of the study groups, as for the tumor size and mass, the data obtained from the study are shown in Table 2.

TABLE 2

| composition version | tumor size reduction % compared to control | tumor mass reduction % compared to control |
|---|---|---|
| Example 1 | 71.8 | 68.3 |
| Example 2 | 68.7 | 65.2 |
| Example 3 | 64.5 | 61.1 |

Thus, the above studies have shown that the composition proposed by the invention increases the expression of p53 protein in cancer cells, which stimulates the process of apoptosis and arrest of the cell cycle G1, and therefore inhibits the proliferation of cancer cells.

1. A pharmaceutical composition based on plant raw materials comprising dry powder of pine needles and dry peels and pits of grapes extract and pharmaceutically acceptable additive, dry extracts of ginger, turmeric, white peony, Rhodiola Rosea, apricot seed, oyster mushroom, green tea, Ginkgo Biloba, white pepper, pomegranate pulp, medlar, dry powder of pomegranate juice and folic acid, in the following ratio of the componentsin w%:

| | |
|---|---|
| dry powder of pine needles and dry peels and pits of grapes extract and pharmaceutically acceptable additive | 6-24 |
| dry extract of ginger | 6-25 |
| dry extract of turmeric | 5-24 |
| dry extract of white peony | 2-20 |
| dry extract of Rhodiola Rosea | 2-20 |
| dry extract of apricot seed | 5-20 |
| dry extract of oyster mushroom | 10-34 |
| dry extract of green tea | 12-35 |
| dry extract of Ginkgo Biloba | 2-25 |
| dry extract of white pepper | 1-15 |
| dry extract of pomegranate pulp | 2-20 |
| extract of medlar | 2-20 |
| dry powder of pomegranate juice | 2-25 |
| folic acid | 3-30 |

.

2. The composition, according to claim 1, wherein it contains components in the following ratio in w%:

| | |
|---|---|
| dry powder of pine needles and dry peels and pits of grapes extract and harmaceutically acceptable additive | 7 |
| dry extract of ginger | 8 |
| dry extract of turmeric | 10 |
| dry extract of white peony | 12 |
| dry extract of Rhodiola Rosea | 10 |
| dry extract of apricot seed | 7 |
| dry extract of oyster mushroom | 10 |
| dry extract of green tea | 12 |
| dry extract of Ginkgo Biloba | 7 |
| dry extract of white pepper | 3 |
| dry extract of pomegranate pulp | 3 |
| extract of medlar | 5 |
| dry powder of pomegranate juice | 3 |
| folic acid | 3 |

.

3. Medicament, comprising the composition according to claims 1-2.

4. Medicament, according to claim 3, wherein it has a form of a capsule.

5. Medicament, according to claim 4, wherein it comprises the composition, according to claims 1-2, in amount of 400-480 mg.

6. Medicament, according to claim 5, wherein it comprises the composition, according to claims 1-2, in amount of 450 mg.
